SEQUENCE LISTING (27 sequences)

SEQ ID NO: 1 - DNA, 18 bp, Artificial Sequence
  primer
atagtggacgcgagcaag

SEQ ID NO: 2 - DNA, 12 bp, Artificial Sequence
  primer
agattgaagagt

SEQ ID NO: 3 - DNA, 32 bp, Artificial Sequence
  primer
ttggcgaaatcgctgaaagaacgtttcttttt

SEQ ID NO: 4 - DNA, 18 bp, Artificial Sequence
  primer
tggtggtttgagaactgg

SEQ ID NO: 5 - DNA, 15 bp, Artificial Sequence
  primer
atagtgtcgacgaac

SEQ ID NO: 6 - DNA, 30 bp, Artificial Sequence
  primer
aacaataaacaaaacaaaggccaaagcctc

SEQ ID NO: 7 - DNA, 18 bp, Artificial Sequence
  primer
gttgatttcgccggactc

SEQ ID NO: 8 - DNA, 12 bp, Artificial Sequence
  primer
ttcgcaagccta

SEQ ID NO: 9 - DNA, 25 bp, Artificial Sequence
  primer
tcgcgcaaaaactccgctggcaaca

SEQ ID NO: 10 - DNA, 18 bp, Artificial Sequence
  primer
gtggtggcttgagaactg

SEQ ID NO: 11 - DNA, 14 bp, Artificial Sequence
  primer
gatagcaaaacgat

SEQ ID NO: 12 - DNA, 36 bp, Artificial Sequence
  primer
cgaaacaaacactaaatgattcctcgttcttgctct

SEQ ID NO: 13 - DNA, 17 bp, Artificial Sequence
  primer
gtggacgcgagcaatgc

SEQ ID NO: 14 - DNA, 18 bp, Artificial Sequence
  primer
aatagagcctggcgaaat

SEQ ID NO: 15 - DNA, 20 bp, Artificial Sequence
  primer
cgaagcaaacgatgacatca

SEQ ID NO: 16 - DNA, 16 bp, Artificial Sequence
  primer
ccgccacccacagtct

SEQ ID NO: 17 - DNA, 18 bp, Artificial Sequence
  primer
agcaaagggaaacaccat

SEQ ID NO: 18 - DNA, 18 bp, Artificial Sequence
  primer
gtttacgcgtccaacgga

SEQ ID NO: 19 - DNA, 18 bp, Artificial Sequence
  primer
cgcgagcaaaacaatggt

SEQ ID NO: 20 - DNA, 10 bp, Artificial Sequence
  primer
taacgatcga

SEQ ID NO: 21 - DNA, 35 bp, Artificial Sequence
  primer
aacgaacaatagagttttcgaaatcaacagcaaaa

SEQ ID NO: 22 - DNA, 18 bp, Artificial Sequence
  primer
tggaagacgtcgttggct

SEQ ID NO: 23 - DNA, 11 bp, Artificial Sequence
  primer
ttatcgcgcca

SEQ ID NO: 24 - DNA, 20 bp, Artificial Sequence
  primer
ggcaaaacgcacccaccgca

SEQ ID NO: 25 - DNA, 18 bp, Artificial Sequence
  primer
gggatgctggtgtggaag

SEQ ID NO: 26 - DNA, 12 bp, Artificial Sequence
  primer
agatgctcgcgt

SEQ ID NO: 27 - DNA, 30 bp, Artificial Sequence
  primer
ccactatccagttcaaaccaccacgcgcca

(19) United States
(12) Patent Application Publication
(10) Pub. No.: US 2016/0082054 A1
(43) Pub. Date: Mar. 24, 2016

(54) SYNBIOTIC COMPOSITION FOR INFANTS

(71) Applicant: N.V. NUTRICIA, ZOETERMEER (NL)

(72) Inventors: Gea SPEELMANS, Utrecht (NL); Jan KNOL, Utrecht (NL); Monique HAARMAN, Vriezenveen (NL); Johan GARSSEN, Utrecht (NL)

(73) Assignee: N.V. NUTRICIA, ZOETERMEER (NL)

(21) Appl. No.: 14/846,012

(22) Filed: Sep. 4, 2015

Related U.S. Application Data
(63) Continuation of application No. 10/576,276, filed on Dec. 28, 2006.

(30) Foreign Application Priority Data
Oct. 24, 2003 (EP) 03078374.0

(51) Int. Cl. A61K 35/745; A23L 1/29; C12Q 1/68; A61K 31/702; A61K 31/715; A23L 1/30; A61K 31/7016

(52) U.S. Cl. CPC A61K 35/745; A23L 1/3014; A23L 1/296; A61K 31/7016; A61K 31/702; A61K 31/715; C12Q 1/689; A23V 2002/00; A23Y 2300/29

(57) ABSTRACT

There is provided a preparation comprising Bifidobacterium breve and a mixture of non-digestible carbohydrates for non- or partially breast-fed infants as well as the use thereof for the treatment or prevention of immune disorder in non- or partially breast-fed infants. Also provided herein are sequence primers and probe for the detection of Bifidobacterium species as well as diagnostic kit thereof.

### CROSS-REFERENCE TO RELATED APPLICATIONS

This application is a Continuation of patent application Ser. No. 10/576,276, filed as the National Phase of International Patent Application No. PCT/NL2004/00748, filed Oct. 25, 2004, published as WO 2005/039319 A2, which claims priority to European Application No. 03078374.0, filed Oct. 24, 2003. The contents of these applications are herein incorporated by reference in their entirety.

The instant application contains a Sequence Listing which has been submitted in ASCII format via EFS-WEB and is hereby incorporated by reference in its entirety. Said ASCII copy, created on Sep. 3, 2015, is named sequence.txt and is 6 KB.

### TECHNICAL FIELD OF THE INVENTION

The present invention relates to preparations comprising a probiotic and a prebiotic for infants, in particular for non-breast-fed infants.

### BACKGROUND OF THE INVENTION

Infants are devoid of intestinal flora at birth. As a result of contact with the mother during birth and subsequent breast feeding, the intestinal flora rapidly develops and increases. During the development, the intestinal flora is still immature and its equilibrium is fragile and quickly prone to changes and thus to the occurrence of diseases and affections in the presence of pathogens. Breast-fed infants are known to be less afflicted by infections or diseases than non-breast-fed infants. Hence, breast-fed babies have less gastro-intestinal infections in terms of both incidence and duration, less atopic diseases such as allergy, eczema, allergy induced asthma, and less constipation than non-breast-fed infants.

Generally, the intestinal flora of breast-fed infants is primarily composed of bifidobacteria and lactic acid bacteria. Breast milk contains human milk oligosaccharides (HMO), which are a growth factor for bifidobacteria in the intestine of infants. The flora of formula-fed infants is more diverse and contains in general more Bacteroides, Clostridium and Enterobacteriaceae species. Formula-fed infants have about one-tenth to roughly two-third the number of bifidobacteria of breast-fed infants. Bifidobacteria are considered to be important in maintaining a well-balanced intestinal microbiota and it has been postulated that bifidobacteria have several health-promoting effects, including the prevention and/or treatment of diarrhea and intestinal infections. Furthermore, bifidobacteria have been shown to play a role in the immune system of the host.

The intestinal flora of infants may be modified by nutritional changes in the diet, like consumption of probiotics or prebiotics. As an example of the probiotics approach, EP-A-0,904,784 describes the administration of a mixture of micro-organism strains, including Bifidobacterium strains. However, a problem associated therewith is that the mixture of microbes, while providing some health benefit, may also have a deleterious effect on the still immature intestinal flora of non-breast-fed infants due to its broad spectrum of action. Further, many probiotic supplements have a short shelf-life and contain too low a number of living microorganisms, thereby failing to provide the expected probiotic effects.

Prebiotics are defined as non-digestible food ingredients that selectively stimulate the growth and/or activity of one or more bacteria in the colon and thereby beneficially affect the host (Gibson and Roberfroid, J. Nutr. 125:1401-14121995). A preferable way to improve the intestinal flora of bottle-fed babies is to selectively stimulate the bifidobacteria already present in the bottle-fed infant's intestine by specific non-digestible oligosaccharides, i.e. prebiotics. Also, mixtures of oligosaccharides and polysaccharides have been proposed as prebiotics, e.g. in WO 00/08948. One example is the combination of galacto-oligosaccharide with fructopolysaccharides. The bifidobacteria level in infants receiving a formula containing these prebiotics has been shown to be elevated in comparison with a standard formula (see e.g. Moro et. al. J. Pediatr. Gastroenterol. Nutr. 34:291-295, 2002).

The approach up to now was to promote bifidobacteria in general, i.e. on the genus level. The genus Bifidobacterium consists of many different species, which differ in metabolism, enzyme activity, oligo- and polysaccharide utilisation, cell wall composition, and interaction with the host's immune system. It therefore can be expected that not every species of Bifidobacterium has the same functional effect on the infant. Examples of different Bifidobacterium species are B. longum, B. breve, B. infantis, B. adolescentis, B. bifidum, B. animalis, and B. dentium. B. adolescentis is more prevalent in the flora of adults, and is less common in faeces of healthy infants and babies. B. animalis/B. lactis is not naturally occurring in humans, and B. dentium is a pathogenic bacterium. In healthy infants the bifidobacterial flora is mainly composed of Bifidobacterium infantis, B. breve and B. longum. Kalliomaki et. al. (Curr Opin Allergy Clin Immunol. 2003 February; 3(1):15-20, and references cited therein), reported that allergic infants harbour an adult-like Bifidobacterium flora whereas a typical infant Bifidobacterium flora was shown in healthy infants, indicating a correlation between the occurrence of certain Bifidobacterium species and the chance of developing allergy. These results indicate that the stimulation of the genus Bifidobacterium in the baby's colon may not be sufficient. It is the aim to achieve a flora in bottle-fed infants that is reminiscent to the flora of breast fed babies on a species level.

For the purpose of the present invention, “breast-fed infants” refers to infants which are exclusively fed with human breast milk. “Non- or partially breast-fed infants” means infants which are not or not exclusively receiving human breast milk. This definition includes those infants which are receiving at least the content of a bottle per day, i.e. at least 80 ml of formula milk per day, the rest, if any, of the nutrition being provided from solid nutrition or liquid nutrition such as breast milk, i.e. partly-breast-fed infants.

### SUMMARY OF THE INVENTION

It was been found that the increase in the level of Bifidobacterium using mixtures of non-digestible carbohydrates also regulates the Bifidobacterium population to a more infant-like population, i.e. low in B. catenulatum, B. pseudocatenulatum and B. adolescentis, whereas infants fed with a standard formula exhibit a more adult-like flora, that is more predominant in B. catenulatum, B. pseudocatenulatum and B. adolescentis. It was also found that the Bifidobacterium population in such prebiotic-fed infants was still deficient in one particular microorganism, namely Bifidobacterium breve.

Accordingly, in one aspect of the invention, there is provided a preparation comprising Bifidobacterium breve and a mixture of non-digestible carbohydrate prebiotics. It was found that such a preparation is beneficial and very suitable for regulating the Bifidibacterium population on a species level in the gastro-intestinal tract of infants. Furthermore, it was surprisingly found that addition of other Bifidobacterium species than B. breve species is not necessary, as they are sufficiently regulated by the preparation as such.

In another aspect of the invention, there is provided a preparation comprising Bifido-bacterium breve and a mixture of non-digestible carbohydrate prebiotics, wherein the mixture of non-digestible carbohydrate contains at least two different, substantially soluble carbohydrate components A and B.

In another aspect of the invention, there is provided the use of the preparation for non- or partially breast-fed infants.

In a further aspect of the invention, there is provided the use of the preparation for the manufacture of a composition for the regulation of the Bifidobacterium species population in the gastro-intestinal tract of non- or partially breast-fed infants.

In a further aspect of the invention, there is provided the use of the preparation for the manufacture of a composition for the prevention or treatment of an immune condition.

In a further aspect of the invention, there is provided the use of a carbohydrate mixture for regulating the population of Bifidobacterium catenulatum, B. pseudocatenulatum and/or Bifidobacterium adolescentis in the gastro-intestinal tract of non- or partially breast-fed infants.

In a still further aspect of the invention, there is provided a method of species-specifically detecting and quantitatively assaying species of the genus Bifidobacterium found in human, particularly human infants, as well as a diagnostic kit for the detection and quantification of Bifidobacterium species.

### BRIEF DESCRIPTION OF THE DRAWINGS

FIG. 1, shows the amounts of each Bifidobacterium species in a culture mix as well as the total amount of Bifidobacterium species in the mix.

In FIG. 2 is a plot of airway hyperresponsiveness as relative PenH (enhanced pause) versus the metacholine concentration for mice receiving a combination of B. Breve+a mixture of GOS/FOS and a control group of mice receiving saline instead. The plotted values of relative PenH are obtained after subtraction of the blank values obtained for mice not ovalbumin-sensitised and normalisation to the value obtained for the control group at the highest concentration of metacholine.

### DETAILED DESCRIPTION OF THE INVENTION

### Preparation

1) Bifidobacterium breve

Bifidobacterium breve is an essential ingredient of the present invention. This bacterium has been found by the Applicant's method of detection as being present in limited quantities in non-breast-fed infants. Accordingly, the administration of this bacterium with the carbohydrate mixture enables the normalisation of the Bifidobacterium species population to a level equivalent to that present in the gastrointestinal tract of breast-fed infants.

Preferred Bifidobacterium breve strains are those selected from isolates from the faeces of healthy breast-fed infants. Typically, these are commercially available from producers of lactic acid bacteria, but they can also be directly isolated from faeces, identified, characterised and produced. Examples of commercially available B. breve are B. breve Bb-03 from Rhodia, B. breve MV-16 from Morinaga, and B. breve from Institut Rosell, Lallemand, but B. breve can also be obtained from culture collections such as DSM 20091, and LMG 11613.

The amount of B. breve in the preparation of the invention can be based on the total amount of soluble non-digestible carbohydrates, and is preferably from 10⁷ to 10¹¹, more preferably from 10⁸ to 10¹⁰ cfu of the bacteria per g of the total of these carbohydrates. When the preparation is used as a supplement, the Bifidobacterium breve is most preferably present in the supplement in an amount of from 1×10⁶ to 1.5×10¹¹ cfu/g, preferably from 3×10⁷ to 5×10¹⁰ cfu/g, more preferably from 5×10⁸ to 1×10¹⁰ cfu/g. When the preparation is used as a (complete) infant nutrition, the B. breve is most preferably present in the nutrition in an amount of from 1×10⁴ to 1×10¹⁰ cfu/g, preferably from 5×10⁶ to 3×10⁹ cfu/g, more preferably from 1×10⁷ to 5×10⁸ cfu per g of the infant nutrition. These concentration are chosen in such a way that the daily dose is about 1×10⁶ to 1.5×10¹¹ cfu/g, preferably from 3×10⁷ to 5×10¹⁰ cfu/g, more preferably from 5×10⁸ to 1×10¹⁰ cfu/g.

### 2) Mixture of Non-Digestible Carbohydrate Prebiotics

A mixture of non-digestible carbohydrate prebiotics is also an essential element of the invention. By “non-digestible”, it is meant that that the carbohydrates remain undigested in the gastrointestinal tract and reach the large intestine unresorbed.

For the purpose of the invention, the mixture of non-digestible carbohydrates contains at least two different, essentially soluble carbohydrate components A and B, which remain undigested in the gastrointestinal tract and reach the large intestine unresorbed. The carbohydrate mixtures according to the present invention may also consist exclusively of these two carbohydrate components A and B.

In the mixture of at least two non-digestible soluble carbohydrate components A and B, the carbohydrate component A is present in an amount of from 5 to 95% by weight of the sum of carbohydrate components A and B. Furthermore, at least 50%, preferably at least 75%, of the total non-digestible soluble carbohydrates of components A and B is selected from disaccharides to eicosasaccharides (polysaccharides having 20 monosaccharide units); the remainder may be non-digestible monosaccharides and non-digestible polysaccharides which are longer than 20 units. It is also preferred that more than 95%, preferably more than 98% of the total soluble non-digestible carbohydrates has a chain length of no more than 100 units. Where percentages and averages are mentioned in this description, percentages and averages by weight are meant, unless it is evident that another basis is meant or when otherwise specified.

The carbohydrates of components may differ in three aspects:

(i) in the (average) number of monosaccharide units of the carbohydrate, component A having an average chain length which is at least 5 monosaccharide units lower than the average chain length of component B; this means that if the carbohydrates of A and B have the same structural units, i.e. they form a mixture of homologues differing only in chain length, the distribution of the homologues must have two maximums, one maximum being below 7, and one above 7, the two maximums being at least 5 units apart; the carbohydrates up to 6 units (hexasaccharides) are then part of component A, and the carbohydrates from 7 units (heptasaccharides) onwards are part of component B;
(ii) in the structure of the monosaccharide units of the carbohydrate, component A being built up from different structural units from component B; where A and/or B are built up from repeating combinations of different monosaccharides units, for example in the case of galactomannans and arabinogalactans, at least 50% of the monosaccharide units of the two components should be different (in the above example either or both should have less than 50% anhydrogalactose units);
(iii) both, i.e. components A and B differ in (average) chain length and in structure; this embodiment is preferred.

Preferably, component A is selected from indigestible monosaccharides up to hexasaccharides of the same carbohydrate structure, and component B is selected from heptasaccharides and higher polysaccharides of the same carbohydrate structure. Carbohydrate component A thereby consists of at least one non-digestible monosaccharide or at least one non-digestible oligosaccharide. With oligosaccharides it is understood those comprising 2 up to and including 6 monosaccharide units. Carbohydrate component A may also, and preferably, be formed by a mixture of two or more of the mentioned saccharides. It may therefore be comprised of any number of various monosaccharides and/or oligosaccharides of that kind, i.e. of the same structure.

According to this preferred embodiment, carbohydrate component B consists of at least one polysaccharide comprising 7 or more monosaccharide units. With polysaccharides it is understood those starting from heptasaccharide (e.g. heptasaccharide, octasaccharide, nonasaccharide, decasaccharide, etc.). There is no specific upper limit to the chain length of polysaccharides, and they may be as long as several hundreds or even thousands of monosaccharide units. However, chain lengths of more than 100 (about 16 kD), and especially those of more than 700 (about 100 Id)) are less preferred according to the invention. Preferably, component B does not contain more than 5% or even not more than 2% of homologues having more than 100 monosaccharide units. Carbohydrate component B may also be comprised of only one polysaccharide of that kind or, preferably, of two or more polysaccharides of different length of that kind, i.e. of the same structure.

Carbohydrate component A represents up to 95 wt % of the sum of carbohydrate component A and carbohydrate component B (A+B=100 wt %). Carbohydrate component B represents 5 to 95 wt % of the sum of carbohydrate component A and carbohydrate component B. According to a preferred embodiment, component A constitutes 95 to 60 wt %, more preferably 95 to 80 wt. % and in particular 95 to 90 wt %, and component B 5 to 40 wt %, more preferably 5 to 20 wt. % and in particular 5 to 10 wt % of the carbohydrates present in toto, with A+B=100 wt %.

As soluble carbohydrates in the sense of the present invention are understood those that are at least 50% soluble, according to a method described by L. Prosky et al, J. Assoc. Anal. Chem 71: 1017-1023, 1988.

At least 80 wt % of the carbohydrates or saccharides out of the sum of carbohydrate component A and B thereby have a prebiotic effect. Preferably, at least 80 wt % of the carbohydrates belonging to carbohydrate component A, and also at least 80 wt % of those belonging to carbohydrate component B, have a prebiotic effect. In other words, preferably at least 80 wt % each of the carbohydrates or saccharides out of carbohydrate components A and B, are intended to reach the large intestine in an undigested (hence not resorbable in the small intestine) manner. In other words, these carbohydrates or saccharides of carbohydrate components A and B in the gastrointestinal tract are neither resorbed and digested in the stomach nor in the small intestine, but reach the large intestine as such.

With a prebiotically active carbohydrate according to the present invention it is understood a carbohydrate, which reaches the large intestine undigested (hence not resorbable in the small intestine), and there, it selectively encourages the growth and/or the activity of one or of a restricted number of bacterial species in the intestine, and consequently promotes health. This prebiotic effect of such carbohydrates and their specific mechanisms are described in detail in “G. F. Gibson & M. B. Roberfroid, J. Nutr. 1995; 125: 1401-1412”, whereto explicit reference is made herewith, and of which the disclosure is included in the present document.

The proportion of the non-prebiotically active carbohydrates or saccharides of carbohydrate components A and B therewith amounts to a maximum of 20 wt %. These carbohydrates or saccharides refer to those which are actually soluble but can be excreted in an undigested form. These carbohydrates can exercise a physical effect in that they increase, for example, the volume of the faeces or prompt a water adsorption.

For the assessment of the proportion determining the carbohydrate components A and B in a dietary or pharmaceutical product, the following steps are carried out. In a first stage, all soluble carbohydrates are extracted from the product by means of water. Fats and proteins are removed from the extract. In a second stage, the soluble carbohydrates or the extract, respectively, are digested by means of human enzymes, e.g. human amylase, human pancreatic juice or small intestine ciliated border preparations. The yield of non-digested carbohydrates (except for the in vivo resorbable monosaccharides obtained in this in vitro experiment), constitutes the two carbohydrate components A and B. 80% thereof are supposed to be prebiotically active.

Hence, the carbohydrate mixtures to be used in the preparation of the invention are those, wherein the carbohydrates, which are soluble and undigested in the sense described above, fulfil the herein specified criteria and constitute the carbohydrate components A and B.

Carbohydrate component A may, for example, consist of one or more of the following carbohydrates: β-galacto-oligosaccharides, α-galacto-oligosaccharides, fructo-oligosaccharides, inulo-oligosaccharides, fuco-oligosaccharides, manno-oligosaccharides, xylo-oligosaccharides, sialyl-oligosaccharides, N-glycoprotein oligosaccharides, O-glyco-protein oligosaccharides, glycolipid oligosaccharides, cello-oligosaccharides, chitosan-oligosaccharides, chitin-oligosaccharides, galacturono-oligosaccharides, glucurono-oligosaccharides, β-glucan (e.g. 1,3-) oligosaccharides, arabinoxylo-oligosaccharides, arabino-galacto-oligosaccharides, xylogluco-oligosaccharides, galactomanno-oligosaccharides, rhamno-oligosaccharides, soy oligosaccharides (stachyose, raffinose, verbascose), and lacto-N-neotetraose. or Carbohydrate component B may, for example, be formed of one or more of the

following carbohydrates or saccharides: fruct(os)anes including inulins, galactans, fucoidans, arabinans, xylans, xanthans, β-glucans, indigestible polydextrose, indigestible maltodextrin, galacturonans, N-glycans, O-glycans, hyaluronic acids, chondroitins, xyloglucans, arabinogalactans, arabic gum, alginates, carrageenanes, galactomannans, glucomannans, arabinoxylanes, glycolipid glycans, glycoprotein glycans, proteoglycans, soy polysaccharides. It is to be noted that digestible carbohydrates are not part of the components A and B. Thus, glucose, fructose, galactose, sucrose, lactose, maltose and the maltodextrins do not count in these components, even if they are the lower homologues of e.g. galacto-oligosaccharides, fructo-oligosaccharides (inulin) and the like. Non-digestible carbohydrates of the invention, as a rule, do not have a large proportion of glucose units linked at the alpha 1,4 and/or alpha 1,6 position as in starch derivatives, as such carbohydrates will be digestible. However, certain starch-type polysaccharides and maltodextrins have been made indigestible or “resistant” by physical or enzymatic means; such oligo- and polysaccharides are included according to the invention, as long as they are sufficiently soluble.

By means of a selective combination of oligosaccharides and polysaccharides, and consequently the simultaneous presence of carbohydrate components A and B, the health-promoting microorganisms in the large intestine may be promoted and/or pathogenic microorganisms may be suppressed by an essentially higher efficiency than would be the case with only one of said carbohydrate components. Thus, it is possible with the administration of the carbohydrate combination, to achieve a very rapid restitution of a normal large intestinal flora, to maintain the same or to prophylactically prevent an alteration of the intestinal flora during situations of stress, and thus to influence the bacterial colonisation of the large intestine in a way, which is more efficient than the one with the previously used carbohydrates.

According to a preferred embodiment, at least 80 wt % of carbohydrate component A as well as of carbohydrate component B consist of carbohydrates, which are bifidogenic and/or which promote lactic acid bacteria. Due to such a combination of oligosaccharides and polysaccharides having said properties, the growth of the lactic acid bacteria may surprisingly be promoted in an essentially stronger manner than would be the case with oligosaccharides or polysaccharides alone. Not only lactic acid bacteria are thereby promoted, which are naturally present in the intestine, but also the growth of those is promoted—optionally even in a selective manner—which are introduced exogenously. Apart from this indirect action via the bacteria themselves and their metabolites such as organic acids (acetate, lactate, etc.), pH effects and stimulation of colonozytes, direct physical effects such as peristalsis, water content, quantity of faeces, mechanical action upon the intestinal mucosa, are likewise positively influenced.

Thus, the carbohydrate mixtures dispose not only of a nutritive effect but also of a wide spectrum of activities. In addition to the above-described biological effects, the following may also be achieved by means of the inventive mixtures: stabilisation of natural microflora, prevention of pathogenic substances/organisms such as toxins, viruses, bacteria, fungi, transformed cells and parasites from adhering, dissolution of complexes of toxins, viruses, bacteria, fungi and other pathogens having endogenous cells, as well as their elimination from the body, and an acceleration of wound healing.

Thus, the mixtures are suitable for the prophylaxis and/or the treatment of symptoms or diseases occurring in conjunction with a disturbed intestinal flora, for example, as a consequence of the association or adhesion of the mentioned substances and organisms with or on epithelia or other endogenous cells.

Carbohydrate mixtures have found to be particularly efficient, when the carbohydrate components A have a different structure than the carbohydrate components B. This different structure may, for example, concern the monosaccharide composition when, for example, fructans are used on the one hand, and galactans on the other hand. This different structure may likewise concern the glycosidic bonding (e.g. α-galacto oligosaccharides versus β-galacto oligosaccharides or α-glucans (starch) versus β-glucans (cellulose)). The monomer composition, as well as the glycoside bonding may have an influence on the chemical behaviour (e.g. solubility) or on the physiological behaviour (e.g. digestibility).

Carbohydrates of the same structure are understood to be homologues which may differ in chain length, but which are composed of the same monosaccharides unit or combination of monosaccharide units. In general, the next homologue will differ from the previous one by the addition of one of the monosaccharide units as present in the previous one. Nevertheless, a single unit, usually a terminal one, may be different, as for example in certain fructans, which contain a chain of (anhydro)fructose units terminated with a glucose unit.

It is preferred that the chain length of the polysaccharide of component B, or the weight-average chain length in case of a mixture of polysaccharides, is at least three units, preferably at least five units longer than the chain length of the oligosaccharide of component A or the weight-average of a mixture of oligosaccharides. Preferably, the average chain length of the oligosaccharides A is between 2 and 6 units, and the average chain length of the polysaccharides B is between 7 and 30, more preferably between 8 and 20. Where both oligosaccharides and polysaccharides of the same structure are present, the carbohydrates of this structure are considered as component A when the weight-average chain length is below 6.5 and the individual members having a chain length of 7 and higher are not counted with the component A; on the other hand, they are considered as component B when the weight-average chain length is above 6.5 and then the individual members having a chain length of 6 and lower are not counted with the component B. Where both oligosaccharides and polysaccharides of the same structure are present in the absence of saccharides of another structure, there should be two maximums at either side of 7 units, or otherwise, the requirement of two different carbohydrate components is not met, as explained above.

The core of the mixtures may inter alia be seen in that carbohydrates of With an administration of mixtures of carbohydrates of different sizes and/or different “classes” or “structures”, a synergistic effect may occur relative to the prebiotic effects of the separate substance groups A and B.

The carbohydrates of component A may belong to one substance class alone but may also be formed out of several classes (for example A: galacto-oligosaccharides plus fuco-oligosaccharides), whereas the carbohydrates of component B may equally originate from one substance class and also from several substance classes (for example B: inulins plus xylans).

A preferred carbohydrate mixture is composed of galacto-oligosaccharide and inulin. Particularly efficient mixtures are those wherein at least 60 wt %, preferably 80 to 100 wt % of carbohydrate components A belong to the group of galacto-oligosaccharides. Also preferred are mixtures wherein at least 60 wt %, preferably 80 to 100 wt % of the carbohydrate components B belong to the group of fructo-polysaccharides. For the production of the carbohydrate mixtures, carbohydrates and carbohydrate mixtures presently known and used in particular for the production of foods or food products can be used. It is also possible to use raw materials previously modified in a technical way. The preparation of the mixtures may thereby ensue by means of a simple blending of the correspondingly selected carbohydrates or oligosaccharides with polysaccharides or the carbohydrate mixtures. The initial components must thereby be so mixed with one another that the parameters are respected with the finished mixtures.

As raw materials may be used reserve carbohydrates (fructans, galacto-oligosaccharides from legumes, fucoidan, α-glucane, laminarin, carrageenan, mannans, galactomannans, agar), natural gum, N-glycosidic bonded carbohydrates of glycoproteins, O-glycosidic bonded carbohydrates of glycoproteins, glycans of glycolipids, enzymatically prepared carbohydrates (galacto-oligosaccharides, gluco-oligosaccharides, fructo-oligosaccharides, xylo-oligosaccharides), bacterial carbohydrates (such as xanthans), as well as oligosaccharides (galacto-oligosaccharides, gluco-oligosaccharides (from a 1-2 and a 1-3 glucose residues), xylo-oligosaccharides), as well as skeletal carbohydrates such as celluloses, hemicelluloses (arabinans, galactans), pectins and chitins may be used. The substances should preferably be of food-grade (cf. Complex Carbohydrates in Foods, British Nutrition Foundation; Chapman & Hall, London 1990).

It is also possible to carry out an enzymatic modification of the raw materials by means of hydrolases (e.g. glycosidases, transglycosidases and lipases), transferases, isomerases (e.g. aldolases and ketolases), oxidoreductases (e.g. oxidases) and reductases (e.g. glucosedehydrogenases), lyases (e.g. polysaccharide lyases) and ligases of the raw materials and products. Moreover, it is possible to carry out a technical modification of the raw materials and products, namely by means of pressure (e.g. extrusion), temperature (e.g. caramelisation), organic syntheses, organic modification (e.g. carboxymethylation and peracetylation), acid and/or alkaline hydrolysis and fractionation (e.g. depending on size and/or physico-chemical parameters such as charge and hydrophobicity) or combinations of modifications.

The carbohydrate mixtures thereby are essentially composed of the monosaccharides listed hereinafter and of the oligosaccharides and polysaccharides composed thereof: D-glucose, D-fructose, D-galactose, D-mannose, L-fucose, D-N-acetylglucosamine, D-N-acetylgalactosamine, D-xylose, L-rhamnose, D-arabinose, D-allose, D-talose, L-idose, D-ribose, as well as monosaccharides comprising carboxyl groups such as D-galacturonic acid, D-glucuronic acid, D-mannuronic acid and/or the methylated forms thereof such as N-acetylneuraminic acid, N-glycolylneuraminic acid and/or O-acetylated forms thereof. Moreover, these monomers and the higher units based thereon can be modified by means of —OSO₃H groups and/or —OPO₃H groups.

Non-digestible carbohydrates according to the present invention are typically administered at a daily dose of 0.5 to 30 g, preferably 2 to 15 g, more preferably 3 to 9 g. One preferred mode of administration of the preparation is as a supplement. The supplement is suited for infants which are non-breast-fed or partly breast-fed, including non- or partly breast-fed prematurely born babies and non- or partly-breast-fed maturely born babies.

The preparation may also be used as an infant nutrition. In this case, the invention infant nutrition further comprises one or more ingredients selected from digestible carbohydrate, a lipid source, protein source, and mixtures thereof.

### 3) Other Components

Apart from the carbohydrate components A and B, other carbohydrates may be present as well. Amongst those are 1) the digestible carbohydrates, which are digestible as described above, and 2) the insoluble carbohydrates, which are resorbable/digestible or even not resorbable/digestible. Typical insoluble non-digestible carbohydrates for use in the infant nutrition supplement are soy polysaccharides, and resistant starch, cellulose and hemicellulose; more preferably they are selected from soy polysaccharides and resistant starch.

Typical soluble and digestible carbohydrate for use in the infant nutrition supplement are selected from maltodextrins, starch, lactose, maltose, glucose, fructose, and sucrose and other mono- and disaccharides, and are more preferably selected from maltodextrin, lactose, maltose, glucose, fructose, sucrose, and mixtures thereof.

These carbohydrates enumerated sub 1) and 2), may be present as such in any arbitrary quantity in addition to the carbohydrate components A and B, in each case depending on the desired final product. Preferably, the insoluble carbohydrates constitute 0 to 10 wt % of the carbohydrate mixtures.

Typical ingredients for use as a lipid source for use in the infant nutrition supplement may be any lipid or fat which is suitable for use in infant formulas. Preferred lipid sources include milk fat, safflower oil, egg yolk lipid, canola oil, olive oil, coconut oil, palm oil, palm kernel oil, palm olein, soybean oil, sunflower oil, fish oil, and microbial fermentation oil containing long-chain polyunsaturated fatty acids. These oils may be in the form of high oleic form such as high oleic sunflower oil and high oleic safflower oil. The lipid source may also be in the form of fractions derived from these oils such as palm olein, medium chain triglycerides (MCT), and esters of fatty acids such as arachidonic acid, linoleic acid, palmitic acid, stearic acid, docosahexaeonic acid, linolenic acid, oleic acid, lauric acid, capric acid, caprylic acid, caproic acid, and the like.

For pre-term formulas, the lipid source preferably contains medium chain triglycerides, preferably in an amount of 15% to 35% by weight of the lipid source.

The lipid source preferably has a molar ratio of n-6 to n-3 fatty acids of 5:1 to 15:1, preferably from 8:1 to 10:1.

When present, it is preferred that the lipid are present at levels of from 20% to 40% by weight of the composition or as 0.8 to 1.5 g/100 kJ in an infant formula.

The proteins that may be utilised in the nutritional products of the invention include any protein or nitrogen source suitable for human consumption. Examples of suitable protein sources for use in infant formula typically include casein, whey, condensed skim milk, non-fat milk, soy, pea, rice, corn, hydrolysed protein, free amino acids, protein sources which contain calcium in a colloidal suspension with the protein and mixtures thereof. It is preferred for use herein that the protein are in hydrolysate form, thereby reducing the risk of allergy in such infant. Commercial protein sources are readily available and known to one practicing the art.

Typically, in the milk-based infant formula hydrolysates 100% hydrolysed whey protein from cow's milk is present. In other milk-based infant formulae the ratio of casein/whey typically is between 1.8:0.3-3.0.

When present, it is preferred that the protein source is present at a levels of from 9% to 19% by weight of the composition. When used as an infant formula, the protein source is preferably present in an amount of from 0.45 to 1.0 g/100 kJ.

A nutritionally complete formula preferably contains all vitamins and minerals understood to be essential in the daily diet and in nutritionally significant amounts. Minimum requirements have been established for certain vitamins and minerals. Examples of minerals, vitamins and other nutrients optionally present in the infant formula include vitamin A, vitamin B, vitamin B2, vitamin B6, vitamin B12, vitamin E, vitamin K, vitamin C, vitamin D, folic acid, inositol, niacin, biotin, pantothenic acid, choline, calcium, phosphorous iodine, iron, magnesium, copper, zinc, manganese, chloride, potassium, sodium selenium, chromium, molybdenum, taurine, and L-carnitine. Minerals are usually added in salt form. The presence and amounts of specific minerals and other vitamins will vary depending on the intended infant population.

If necessary, the infant formula may contain emulsifiers and stabilisers such as soy lecithin, citric acid, esters of mono and di-glycerides, and the like. This is especially provided if the formula is to be provided in liquid form.

The infant formula may optionally contain other substances which may have a beneficial effect such as (non-carbohydrate) fibres, lactoferrin, immunoglobulins, nucleotides, nucleosides, and the like.

### Applications

The preparations according to the invention have been found to be particularly useful in the normalisation of the Bifidobacterium population according to the species distribution in breast-fed infants, considered as “standard”, in the gastro-intestinal tract of infants which were non- or partly breast-fed, in particular those which are prematurely born babies, maturely born babies, as well as infants which are in the adaptation period to solid food. The preparation of the invention is also suitable for infants changing from breast to bottle feeding.

Accordingly, there is provided the use of the invention preparation or composition for the manufacture of a composition for the normalisation of the Bifidobacterium species population in the gastro-intestinal tract of non- or partly breast-fed infants The preparations of the invention have also been found particularly useful for the prevention or treatment of an immune condition. This immune condition is believed to be the result of the difference in the composition of the Bifidobacterium species in the gastrointestinal tract of these non- or partly breast-fed infants when compared to that of breast-fed infants. Typically, such immune conditions include conditions selected from allergy, atopic dermatitis, eczema, asthma, atopy, allergic rhinitis, food hypersensitivity, diapers rashes, diarrhoea, and mixtures thereof.

Accordingly, the invention provides the use of the preparation for the prevention or treatment of one or more an immune conditions, preferably selected from allergy, atopic dermatitis, eczema, asthma, and diapers rashes. Also (bacterial) diarrhoea and especially viral diarrhoea can be treated with the preparation of the invention. Also provided herein is the use of the preparation for the prevention and/or treatment of energy malabsorption.

Advantageously, the preparation has been found beneficial for inhibiting the infiltration of eosinophils, neutrophils and mononuclear cells in allergic lesions, and/or inhibiting the Th2 type immune response and/or stimulating the Th1 mediated immune response. Accordingly, there is provided the use of the invention preparation or composition as defined herein for the manufacture of a composition for inhibiting the infiltration of eosinophils, neutrophils and mononuclear cells in allergic lesions, inhibiting the Th2 type immune response and/or stimulating the Th1 mediated immune response.

The invention also provides the use of the carbohydrate mixture as described above for regulating the population of certain Bifidobacterium species other than B. breve, in particular for decreasing the relative amounts of Bifidobacterium catenulatum, B. pseudo-catenulatum and/or B. adolescentis.

### Probe Development and Diagnostic Kit

Also provided herein is a method for quantifying Bifidobacterium species, especially those found in humans, i.e. Bifidobacterium catenulatum and B. pseudocatenulatum, B. adolescentis, B. breve, B. longum, B. bifidum, B. angulatum, B. infantis, and B. dentium, using species specific oligonucleotide primes and probes.

These primers and probes can be used to identify bifidobacteria and bifidobacterial species via FISH, PCR, DGGE, TGGE, dot blot hybridisation and real time PCR methods. All these techniques have in common that it involves a hybridisation step with nucleotides. It is especially the purpose to determine the quantities of species of bifidobacteria by real time PCR.

Each of the sequences described below may have additional bases bonded to the 5′- or 3′-terminal thereof as long as it functions as a probe.

These oligonucleotides can be prepared by conventional means for chemical synthesis, for example by an automated DNA synthesiser. DNA fragments containing the above-mentioned sequences can be prepared by enzymatic cleavage of genes from the corresponding Bifidobacterium species.

For the purpose of the present invention, the development of primers and probes specific to the Bifidobacteria species for use in the 5′ nuclease assay was as follows: Duplex 5′ nuclease assays were developed for Bifidobacterium adolescentis, B. angulatum, B. bifidum, B. breve, B. catenulatum, B. dentium, B. longum and B. infantis in relation to all bifidobacteria. We developed The 5′ nuclease assays on the intergenic spacer of 16S-23S rDNA instead of the 16S rDNA gene, which is normally used for the phylogenetic analyses and specific detection of bacteria. The choice for the intergenic spacer greatly depended on the fact that contamination and sensitivity issues were described for Real Time PCR when 16S rDNA was used. Furthermore, a large similarity between the 16S rDNA sequences of the different Bifidobacterium species was shown (Leblond-Bourget et. al. 1996), which made it almost impossible to develop primer and probe sets specific for the different Bifidobacterium species. Surprisingly, these problems could be avoided by using the intergenic spacer region.

For the development of primers and probes the different sequences of the 16S-23S intergenic spacer region of the different Bifidobacterium species (B. adolescentis [U09511 U09512 (1), U09513 (1) and U09514 (1)]^{a}, B. angulatum [U09515 (1)]^{a}, B. animalis [AY225132 (2), L36967 (1) and U09858 (1)]^{a}, B. asteroides [U09516 (1)]^{a}, B. breve [AJ245850 (3), U09518 (1), U09519 (1), U09520 (1) and U09521 (1)]^{a}, B. bifidum [U09517 (1), U09831 (1)]^{a}, B. catenulatum [U09522 (1)]^{a}, B. choerinum [L36968 (1)]^{a}, B. coryneforme [U09523 (1)]^{a}, B. cuniculi [U09790 (1)]^{a}, B. dentium [U10434 (1)]^{a}, B. indicum [U09791 (1)]^{a}, B. infantis [AJ245851 (3), U09525 (1), U09527 (1) and U09792 (1)]^{a}, B. longum [AJ245849 (3), U09832 (1)]^{a}, B. pseudolongum [U09524 (1), U09879 (1)]^{a}, B. magnum [U09878 (1)]^{a}, B. thermophilum [U09528 M]^{a}) were retrieved from Genbank, EMBL and DDBJ databases. All retrieved sequences were aligned using DNASIS for Windows V2.5 (Hitachi Software Engineering Co., Ltd., Wembley, UK). (^{a}=accession codes, 1=Leblond-Bourget, N., H. Philippe, I. Mangin, B. Decaris. 1996. 16S rRNA and 16S to 23S internal transcribed spacer sequence analyses reveal inter- and intraspecific Bifidobacterium phylogeny. Int. J. Syst. Bacteriol. 46:102-111, 2=Ventura, M., and R. Zink. 2002. “Rapid identification, differentiation, and proposed new taxonomic classification of Bifidobacterium lactis.” Appl. Environ. Microbiol. 68:6429-6434, 3=Brigidi, P., B. Vitali, E. Swennen, L. Altomare, M. Rossi, and D. Matteuzzi. 2000. “Specific detection of Bifidobacterium strains in a pharmaceutical probiotic product and in human feces by polymerase chain reaction.” Syst. Appl. Microbiol. 23:391-399.), The overall conserved regions of the sequences were used to design primers and probes for all Bifidobacterium species. Conserved regions in the sequences of the different kind of subspecies, which showed little homology with other species were used to design primers and probes for respectively B. adolescentis, B. angulatum, B. breve, B. bifidum, B. catenulatum (including B. pseudocatenulatum), B. dentium, B. infantis and B. longum (including B. pseudolongum due to a great homology in sequence between these two species).

The primers and TaqMan MGB probes were designed with help of Primer Express 1.5a (Applied Biosystems, Nieuwerkerk a/d Ussel, NL). We applied the following criteria: The probe and primers should have a GC content of 30 to 80% and runs of more than 3 identically nucleotides (especially for guanidine (G)) should be avoided. The melting temperature (Tm) of the probe should be between 68° C. and 70° C., whereas the primers should have a melting temperature 10° C. below the melting temperature of the probe. Furthermore, no G on the 5′ end of the probe should be present and the strand with more cytosine (C) than G was selected. The last 5 nucleotides at the 3′ end of the primers should have no more than two G and/or C bases. Finally, the amplicon length should be less than 150 base pairs. The designed primers and TaqMan MGB probes are shown in table 1 and were tested on specificity using the Basic Local Alignment Search Tool (BLAST).

The probe designed for the detection of all Bifidobacterium consists of an oligonucleotide with the 5′ reporter dye VIC™ (Applied Biosystems, NL) and the 3′ quencher NFQ-MGB™ (Applied Biosystems, NL) and the probes for the different Bifidobacterium species of oligonucleotides with the 5′ reporter dye 6-carboxy-fluorescein (FAM™) and the 3′ quencher NFQ-MGB™ (Applied Biosystems, NL). For determination of the total bacterial load a broad-range (universal) probe and primer set is used, which is described by Nadkarni, M. A., F. E. Martin, N. A. Jacques, and N. Hunter “Determination of bacterial load by real-time PCR using a broad-range (universal) probe and primers set.” Microbiology 148:257-266 (2002). The universal probe consists of oligonucleotides with the 5′ reporter dye 6-carboxy-fluorescein (FAM™) and the 3′ quencher dye 6-carboxy-tetramethyl-rhodamine (TAMRA™). The designed probes are shown in table 1.

TABLE 1
Designed primers and probes for use in the 5′ nuclease assays

| | Primers & | Sequence | Tm | % | BLAST ID | Amplicon | SEQ |
|---|---|---|---|---|---|---|---|
| Target | Probes | (5′ → 3′) | (° C.) | GC | number | length | ID No |
| B. adolescentis | F_adol_IS | ATA GTG GAC | 59 | 52 | 1015335678- | 71 bp | 1 |
| | | GCG AGC AAG | | | 6465-18906 | | |
| | | AGA | | | | | |
| | R_adol_IS | TTG AAG AGT | 59 | 43 | 1015335740- | | 2 |
| | | TTG GCG AAA | | | 7519-1624 | | |
| | | TCG | | | | | |
| | P_adol_IS | CTG AAA GAA | 69 | 30 | 1015335863- | | 3 |
| | | CGT TTC TTT | | | 95222-17207 | | |
| | | TT^{a} | | | | | |
| B. angulatum | F_angul_IS | TGG TGG TTT | 59 | 46 | 1015336044- | 117 bp | 4 |
| | | GAG AAC TGG | | | 12581-14600 | | |
| | | ATA GTG | | | | | |
| | R_angul_IS | TCG ACG AAC | 59 | 32 | 1015336147- | | 5 |
| | | AAC AAT AAA | | | 14351-29932 | | |
| | | CAA AAC A | | | | | |
| | P_angul_IS | AAG GCC AAA | 70 | 57 | 1015488648- | | 6 |
| | | GCC TC | | | 5575-2104 | | |
| B. bifidum | F_bif_IS | GTT GAT TTC | 60 | 52 | 1015336612- | 105 bp | 7 |
| | | GCC GGA CTC | | | 215666-12828 | | |
| | | TTC | | | | | |
| | R_bif_IS | GCA AGC CTA | 60 | 56 | 1015336668- | | 8 |
| | | | | | 22451-30731 | | |
| | P_bif_IS | TCG CGC AAA | 70 | 56 | 1015336773- | | 9 |
| | | AAC TCC GCT | | | 24053-3416 | | |
| | | GGC AAC A | | | | | |
| B. breve | F_breve_IS | GTG GTG GCT | 59 | 52 | 1015243936- | 118 bp | 10 |
| | | TGA GAA CTG | | | 11550-20833 | | |
| | | GAT AG | | | | | |
| | R_breve_IS | CAA AAC GAT | 58 | 32 | 1015244110- | | 11 |
| | | CGA AAC AAA | | | 13595-29514 | | |
| | | CAC TAA A | | | | | |
| | P_breve_IS | TGA TTC CTC | 69 | 45 | 1015244238- | | 12 |
| | | GTT CTT GCT | | | 15062-16853 | | |
| | | GT | | | | | |
| B. catenulatum | F_cate_IS | GTG GAC GCG | 58 | 65 | 1015335268- | 67 bp | 13 |
| | | AGC AAT GC | | | 99-20718 | | |
| | R_cate_IS | AAT AGA GCC | 58 | 50 | 1015335364- | | 14 |
| | | TGG CGA AAT | | | 1571-12175 | | |
| | | CG | | | | | |
| | P_cate_IS | AAG CAA ACG | 68 | 39 | 1015335455- | | 15 |
| | | ATG ACA TCA | | | 2899-17859 | | |
| B. dentium | F_dent_IS | CCG CCA CCC | 59 | 71 | 1015399643- | 150 bp | 16 |
| | | ACA GTC T | | | 15856-19947 | | |
| | R_dent_IS | AGC AAA GGG | 59 | 41 | 1015399751- | | 17 |
| | | AAA CAC CAT | | | 16991-11210 | | |
| | | GTT T | | | | | |
| | P_dent_IS | ACG CGT CCA | 70 | 64 | 1015399833- | | 18 |
| | | ACG GA | | | 18158-5198 | | |
| B. infantis | F_inf_IS | CGC GAG CAA | 58 | 47 | 1037961234- | 76 bp | 19 |
| | | AAC AAT GGT | | | 06371-14364 | | |
| | | T^{a} | | | | | |
| | R_inf_IS | AAC GAT CGA | 58 | 36 | 1037961263- | | 20 |
| | | AAC GAA CAA | | | 06691-25461 | | |
| | | TAG AGT T | | | | | |
| | P_inf_IS | TTC GAA ATC | 69 | 32 | 1037961294- | | 21 |
| | | AAC AGC AAA | | | 06967-17477 | | |
| | | A^{a} | | | | | |
| B. longum | F_long_IS | TGG AAG ACG | 59 | 50 | 1015323391- | 109 bp | 22 |
| | | TCG TTG GCT | | | 27595-22257 | | |
| | | TT | | | | | |
| | R_long_IS | ATC GCG CCA | 58 | 56 | 1015323469- | | 23 |
| | | GGC AAA A^{a} | | | 28673-23147 | | |
| | P_long_IS | CGC ACC CAC | 68 | 77 | 1015488566- | | 24 |
| | | CGC A | | | 4529-13934 | | |
| All | F_allbif_IS | GGG ATG CTG | 60 | 57 | 1015399960- | 231 bp^{a} | 25 |
| Bifidobacterium | | GTG TGG AAG | | | 19603-31240 | | |
| | | AGA | | | | | |
| | R_allbif_IS | TGC TCG CGT | 60 | 57 | 1015400076- | | 26 |
| | | CCA CTA TCC | | | 20827-17418 | | |
| | | AGT | | | | | |
| | P_allbif_IS | TCA AAC CAC | 70 | 61 | 1015400166- | | 27 |
| | | CAC GCG CCA | | | 21749-18424 | | |

^{a}In these cases some adjustments (more than 3 consecutive nucleotides or an amplicon length greater then 150 bp) were made to the guidelines to find an appropriate primer and probe set.

Labeled preparations are prepared by labeling the oligonucleotide with a detectable marker by conventional means. Labeling markers which may be used include radioisotopes, fluorescent substances, enzymes, biotin and haptens.

Hybridisation between the labeled preparation and a sample can be performed in accordance with known techniques, such as dot blot hybridisation and northern hybridisation. The hybrid which are formed can be confirmed through the detection of the labeled preparation by known means, for example, autoradiography using radioisotopes, enzyme-labeled antibody techniques using enzyme or biotin, and the like.

Further, of these oligonucleotides, the DNA fragments represented by SEQ ID selected from SEQ ID No 1, SEQ ID No 2, SEQ ID No 4, SEQ ID No 5, SEQ ID No 7, SEQ ID No 8, SEQ ID No 10, SEQ ID No 11, SEQ ID No 13, SEQ ID No 14, SEQ ID No 16, SEQ ID No 17, SEQ ID No 19, SEQ ID No 20, SEQ ID No 22, SEQ ID No 23, SEQ ID No 25, SEQ ID No 26 respectively) can be used as a primer in the PCR method for identification of species. More specifically, microbial cells to be identified are subjected to bacteriolysis, and any of the DNA fragments of SEQ ID selected from SEQ ID No 1, SEQ ID No 4, SEQ ID No 7, SEQ ID No 10, SEQ ID No 13, SEQ ID No 16, SEQ ID No 19, SEQ ID No 22, SEQ ID No 25, respectively) and SEQ ID selected from SEQ ID No 2, SEQ ID No 5, SEQ ID No 8, SEQ ID No 11, SEQ ID No 14, SEQ ID No 17, SEQ ID No 20, SEQ ID No 23, SEQ ID No 26, respectively) is added thereto as a primer, followed by treatment with a DNA polymerase. If DNA amplification is observed using electrophoresis, etc., this means that the cells possess a gene portion which corresponds to the DNA fragment used, i.e. the cells are identified to be of the same species as the origin of the DNA fragment primer.

Accordingly, there are provided oligonucleotides comprising SEQ ID selected from SEQ ID No 1, SEQ ID No 2, SEQ ID No 4, SEQ ID No 5, SEQ ID No 7, SEQ ID No 8, SEQ ID No 10, SEQ ID No 11, SEQ ID No 13, SEQ ID No 14, SEQ ID No 16, SEQ ID No 17, SEQ ID No 19, SEQ ID No 20, SEQ ID No 22, SEQ ID No 23, SEQ ID No 25, SEQ ID No 26, and sequences complementary thereto.

Also provided herein are oligonucleotide probe for detection of a nucleic acid target sequence which is characteristic of the species of the genus Bifidobacterium, said probe being selected from:

1)—a labelled oligonucleotide which specifically hybridises to Bifidobacterium adolescentis DNA represented by SEQ ID No 3 or a sequence complementary thereto;
2)—a labelled oligonucleotide which specifically hybridises to Bifidobacterium angulatum DNA represented by SEQ ID No 6 or a sequence complementary thereto;
3)—a labelled oligonucleotide which specifically hybridises to Bifidobacterium bifidum DNA represented by SEQ ID No 9 or a sequence complementary thereto;
4)—a labelled oligonucleotide which specifically hybridises to Bifidobacterium breve DNA represented by SEQ ID No 12 or a sequence complementary thereto;
5)—a labelled oligonucleotide which specifically hybridises to Bifidobacterium catenulatum DNA represented by SEQ ID No 15 or a sequence complementary thereto;
6)—a labelled oligonucleotide which specifically hybridises to Bifidobacterium dentium DNA represented by SEQ ID No 18 or a sequence complementary thereto;
7)—a labelled oligonucleotide which specifically hybridises to Bifidobacterium infantis DNA represented by SEQ ID No 21 or a sequence complementary thereto;
8)—a labelled oligonucleotide which specifically hybridises to Bifidobacterium longum DNA represented by SEQ ID No 24 or a sequence complementary thereto;
9)—a labelled oligonucleotide which specifically hybridises to all Bifidobacterium DNA represented by SEQ ID No 27 or a sequence complementary thereto.

Further provided herein is a method of species-specifically detecting species of the genus Bifidobacterium found in human, particularly human infants, comprising the steps of:

(A) contacting a sample with an oligonucleotide probe in a hybridising solution, wherein said probe is selected from the group consisting of:
1) a labelled oligonucleotide which specifically hybridises to Bifidobacterium adolescentis DNA represented by SEQ ID No 3 or a sequence complementary thereto;
2) a labelled oligonucleotide which specifically hybridises to Bifidobacterium angulatum DNA represented by SEQ ID No 6 or a sequence complementary thereto;
3) a labelled oligonucleotide which specifically hybridises to Bifidobacterium bifidum DNA represented by SEQ ID No 9 or a sequence complementary thereto;
4) a labelled oligonucleotide which specifically hybridises to Bifidobacterium breve DNA represented by SEQ ID No 12 or a sequence complementary thereto;
5) a labelled oligonucleotide which specifically hybridises to Bifidobacterium catenulatum DNA represented by SEQ ID No 15 or a sequence complementary thereto; 6) a labelled oligonucleotide which specifically hybridises to Bifidobacterium dentium DNA represented by SEQ ID No 18 or a sequence complementary thereto;
7) a labelled oligonucleotide which specifically hybridises to Bifidobacterium infantis DNA represented by SEQ ID No 21 or a sequence complementary thereto;
8) a labelled oligonucleotide which specifically hybridises to Bifidobacterium longum DNA represented by SEQ ID No 24 or a sequence complementary thereto;
9) a labelled oligonucleotide which specifically hybridises to all Bifidobacterium DNA represented by SEQ ID No 27 or a sequence complementary thereto, and
(B) determining whether said probe hybridises to nucleic acids in said sample so as to detect whether said species of said genus is present in said sample.

The present invention also encompasses a method of species-specifically detecting species of the genus Bifidobacterium found in human, particularly human infants, comprising the steps of:

a) performing a nucleic acid sequence amplification procedure using a primer set comprising the oligonucleotide primer of SEQ ID No selected from SEQ ID No 1, SEQ ID No 4, SEQ ID No 7, SEQ ID No 10, SEQ ID No 13, SEQ ID No 16, SEQ ID No 19, SEQ ID No 22, SEQ ID No 25, and respectively with the oligonucletide primer of SEQ ID selected from SEQ ID No 2, SEQ ID No 5, SEQ ID No 8, SEQ ID No 11, SEQ ID No 14, SEQ ID No 17, SEQ ID No 20, SEQ ID No 23, SEQ ID No 26; and
b) determining whether the oligonucleotide probe above mentioned hybridises to the nucleic acid target sequence.

The present method is beneficial for the manufacture of a diagnostic kit. Accordingly, a diagnostic kit is herein provided for the detection in a sample of Bifidobacterium species selected from Bifidobacterium adolescentis, B. angulatum, B. bifidum, B. breve, B. catenulatum, B. dentium, B. infantis and B. longum, by means of hybridisation analysis, comprising at least a DNA probe as mentioned above as well as one or more further means required for hybridisation analysis, such as denaturation liquid, a hybridisation liquid, a washing liquid, a solid carrier, a hybridisation vessel and label detecting means.

Also herein provided is a diagnostic kit for the detection in a sample of the above-mentioned Bifidobacterium species by means of PCR analysis, comprising a set of DNA primers as mentioned above as well as one or more further means required for PCR analysis, such as a polymerase, a polymerisation liquid, an oil overlay, a reaction vessel and means for detecting the amplified DNA.

### Example 1

### Validation of the Developed Probes and Primers for Bifidobacteria

The bacterial strains used to validate the assays for the relative quantification of the different Bifidobacterium species are listed in Table 2.

TABLE 2
Bacterial strains and origins used for the
development of the 5′ nuclease assays

| Strain | Origin^{a} | | |
|---|---|---|---|
| Bifidobacterium strains | | | |
| B. adolescentis | ATCC 15703^{T} | ATCC 15705 | |
| B. angulatum | DSM 20098^{T} | | |
| B. animalis | ATCC 25527^{T} | DSM 10140 | |
| B. bifidum | DSM 20456^{T} | NCIMB 8810 | |
| B. boum | ATCC 27917^{T} | | |
| B. breve | ATCC 15700^{T} | DSM 20091 | LMG |
| | | | 11613 |
| B. catenulatum | ATCC 27539^{T} | ATCC 27675 | |
| B. dentium | ATCC 27534^{T} | | |
| B. gallicum | DSM 20093^{T} | | |
| B. gallinarum | ATCC 33777^{T} | | |
| B. infantis | LMG 8811^{T} | | |
| B. inopinatum | DSM 10107^{T} | | |
| B. longum | ATCC 15707^{T} | | |
| B. magnum | ATCC 27540^{T} | | |
| B. pseudocatenulatum | DSM 20438^{T} | | |
| B. pseudolongum | ATCC 25526^{T} | | |
| B. suis | ATCC 27533^{T} | | |
| Other Strains | | | |
| Bacillus cereus | ATCC 11778 | | |
| Bacteroides fragilus | LMG 10263^{T} | | |
| Brevibacterium casei | ATCC 35513^{T} | | |
| Clostridium difficile | ATCC 9689^{T} | | |
| Enterococcus feacalis | DSM 20478^{T} | | |
| Escherichia coli | ATCC 35218 | | |
| Lactobacillus acidophilus | ATCC 4356^{T} | | |
| Lactobacillus brevis | LMG 18022 | | |
| Lactobacillus bulgaricus | ATCC 11842^{T} | | |
| Lactobacillus casei | ATCC 393^{T} | DSM 20011^{T} | |
| Lactobacillus fermentum | DSM 20052^{T} | | |
| Lactobacillus plantarum | DSM 20174^{T} | | |
| Lactobacillus reuteri | LMG 9213^{T} | | |
| Lactobacillus rhamnosus | ATCC 53103 | | |
| Listeria monocytogenes | ATCC 7644 | | |
| Pediococcus acidilactici | DSM 20284^{T} | | |
| Propionibacterium avidum | DSM 4901 | | |
| Pseudomonas aeruginosa | DSM 1117 | | |
| Saccharomyces cerevisiae | DSM 2548 | | |
| Salmonella typhimurum | ATCC 14028 | | |
| Staphylococcus aureus | ATCC 29213 | | |

ATCC: American Type Culture Collection;
DSM: Deutsche Sammlung von Mikroorganismen und Zellkulturen, Germany;
LMG: Laboratory for Microbiology, University of Gent, Belgium;
NCIMB: National Collections of Industrial and Marine Bacteria, UK.

All bifidobacteria strains were cultured in Mann Rogosa Sharp (MRS) broth (Oxoid, Basingstoke, UK) media at 37° C. for 24 hours under anaerobic conditions. The overnight cultures were stored at −20° C. until further processing.

DNA was extracted from bacterial cultures by thawing 5 ml of frozen overnight cultures in ice water. Subsequently, the cultures were centrifuged for 20 minutes at 4000 rpm at 4° C. (Sorvall RT7, Du Pont, Stevenage, UK) to pellet the bacterial cells. The pellets were washed with 1 ml TES (50 mM Tris-HCl [pH 8.0], 5 mM EDTA, 50 mM NaCl), followed by a centrifugation step of 10 minutes at 4000 rpm at 4° C. Supernatants were removed and the pellet were resuspended in 1 ml of THMS (30 mM Tris-HCl [pH 8.0], 3 mM MgCl₂, 25% (w/v) sucrose). After transfer of the suspensions into a two ml eppendorf tube, 200 μl lysozyme (0.1 g/ml; Sigma Aldrich Chemie, Steinheim, DE) and 40 μl mutanolysine (1 mg/ml; Sigma Aldrich Chemie, DE) was added and incubated for 30 minutes at 37° C. Subsequently, the solutions were centrifuged for 5 minutes at 10000 rpm at 4° C. (Sigma 1-15, Sigma Laborzentrifugen GmbH, Osterode am Harz, DE). Supernatants were removed and the pellets were resuspended in 100 μl THMS, whereto 400 μl TES (including 0.5% SDS) and 7.5 μl of Proteinase K (20 mg/ml; Boehringer Mannheim GmbH, Mannheim, DE) were added. The mixture was vortexed and incubated for 30 minutes at 65° C. Subsequently, a standard phenol/chloroform extraction was carried out, followed by a treatment with 2.5 μl RNase A (1 mg/ml; Roche Diagnostics, Mannheim, DE) for 30 minutes at 37° C. Subsequently, the DNA was precipitated by storing at −20° C. for at least 30 minutes after addition of 2 volumes ice cold ethanol (96%) and 0.1 volume of 3 M sodium acetate (pH 5.2). Precipitated solutions were centrifuged for 20 minutes at 13000 rpm at 4° C. and the supernatants were washed with 500 μl 70% ethanol, followed by centrifugation at 13000 rpm for 5 minutes at 4° C. Supernatants were discarded and the pellets were air dried at room temperature. The DNA was resuspended in 100 μl sterile milli-Q and stored at −20° C.

Firstly, the specificity of each duplex 5′ nuclease assay was tested by performing a 25 μl amplification of the different strains (see table 2). These 25 μl PCR reactions were performed using 2.5 μl DNA template, 12.5 μl TaqMan Universal Master Mix (Applied Biosystems), 900 nM of each primer and 200 nM of each probe, followed by running the TaqMan Universal Temperature Profile, which consists of 2 minutes at 50° C., 10 minutes at 95° C., followed by 45 cycles of 15 seconds at 95° C. and 60° C. for 1 minute, on the ABI Prism 7700 (Applied Biosystems, Nieuwerkerk a/d IJssel, NL). All of the 5′ nuclease assays were specific for the Bifidobacterium species for which they were developed and the 5′nuclease assay for determination of the total amount of Bifido-bacterium detected all Bifidobacterium species tested, but no other strains like Propioni-bacterium or Lactobacillus. It should be noted that the 5′nuclease assay for B. catenulatum also detects B. pseudocatenulatum. Furthermore, DNAse and RNAse treated samples were tested to assure that no contaminated RNA was detected during the assay.

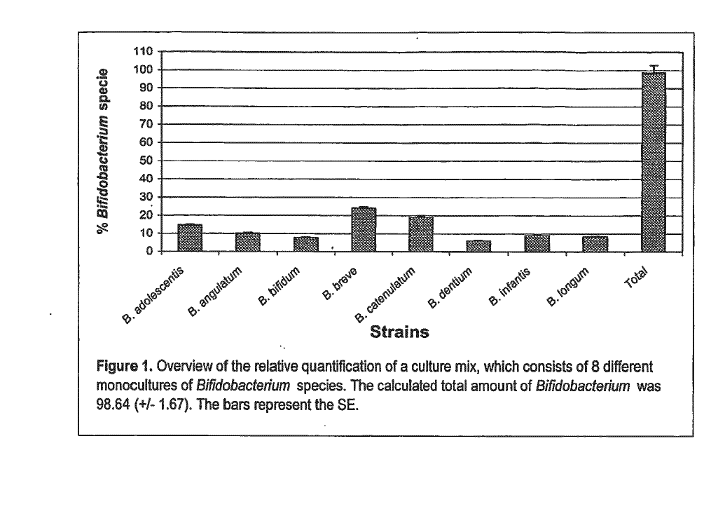

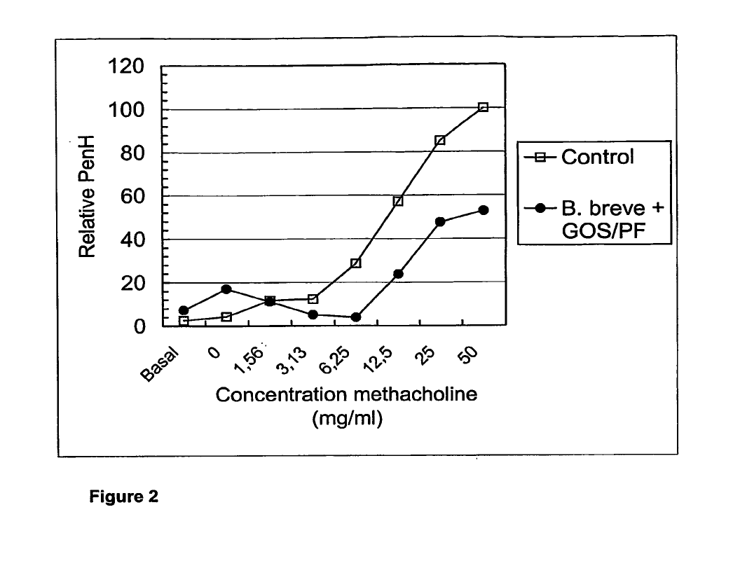

Secondly, a mix of monocultures from B. adolescentis, B. angulatum, B. breve, B. bifidum, B. catenulatum, B. dentium, B. infantis and B. longum was prepared to verify that the total of this mix would sum up to approximately 100%. In that case, competition between the different Bifidobacterium species, which serve as template, can be excluded. This is indeed the case, as be seen in FIG. 1, which shows the determined amounts of each Bifidobacterium species in the mix as well as the total amount of Bifidobacterium species in the mix.

The CV values for reproducibility and repeatability for the different kind of 5′ nuclease assays were determined and can be found in table 3.

TABLE 3
Sensitivity of the 5′ nuclease assays in comparison to “conventional”
PCR and reproducibility and repeatability of the 5′ nuclease assays

| | Sensitivity^{a} | Reproducibility^{b} | Repeatability^{c} |
|---|---|---|---|
| Target | (x) | [CV (%)] | [CV (%)] |
| B. adolescentis | 10,000 | 5.11 | 5.68 |
| B. angulatum | 1000 | 19.48 | 20.92 |
| B. bifidum | 100 | 11.65 | 11.20 |
| B. breve | 100 | 2.06 | 4.08 |
| B. catenulatum | 1000 | 9.42 | 14.83 |
| B. dentium | 100 | 12.65 | 11.35 |
| B. infantis | 1000 | 2.34 | 2.31 |
| B. longum | 10,000 | 9.10 | 8.18 |

^{a}number of times that the 5′ nuclease assay is more sensitive then “conventional” PCR
^{b}reproducibility is determined by testing monocultures (100%) in ten fold and calculation of the CV (%) based on the gained results
^{c}repeatability is determined by testing monocultures (100%) three times in four fold and calculation of the CV (%) based on results gained

The developed 5′ nuclease assays were compared to the conventional qualitative species-specific PCR (using the primers as described by Matsuki, T., K. Watanabe, R. Tanaka, M. Fukuda, and H. Oyaizu. 1999. Distribution of bifidobacterial species in human intestinal microflora examined with 16S rRNA-gene-targeted species-specific primers. Appl. Environ. Microbiol. 65:4506-4512) to determine the sensitivity of the different assays as well as checking for false positive or negative results. Table 3 shows the different sensitivities of the 5′ nuclease assays in relation to the conventional species specific PCR. Table 4 show the final optimal primer and probe concentrations used in the duplex 5′ nuclease assays.

TABLE 4
Optimised final primer and probe concentrations
used in the different duplex 5′ nuclease assays

| | | | Reverse | |
|---|---|---|---|---|
| | | Forward | Primer | Probe |
| Target | 5′ nuclease assay | Primer (nM) | (nM) | (nM) |
| B. adolescentis | B. adolescentis | 300 | 150 | 100 |
| | All Bifidobacterium | 300 | 600 | 100 |
| B. angulatum | B. angulatum | 900 | 900 | 200 |
| | All Bifidobacterium | 300 | 300 | 50 |
| B. bifidum | B. bifidum | 600 | 600 | 200 |
| | All Bifidobacterium | 300 | 300 | 100 |
| B. breve | B. breve | 300 | 300 | 100 |
| | All Bifidobacterium | 450 | 450 | 150 |
| B. catenulatum | B. catenulatum | 300 | 300 | 100 |
| | All Bifidobacterium | 600 | 600 | 100 |
| B. dentium | B. dentium | 900 | 900 | 200 |
| | All Bifidobacterium | 300 | 300 | 50 |
| B. infantis | B. infantis | 300 | 300 | 100 |
| | All Bifidobacterium | 900 | 900 | 100 |
| B. longum | B. longum | 300 | 300 | 100 |
| | All Bifidobacterium | 600 | 600 | 200 |
| All | All Bifidobacterium | 450 | 450 | 100 |
| Bifidobacterium | All bacteria | 900 | 900 | 200 |

### Example 2

### Clinical Trial

The study was a double blind, placebo-controlled multi-center trial with two intervention groups. Fully formula fed infants, aged 28 to 90 days, were recruited from four hospitals in Germany. Infants were included in the study if they had a birth weight between 2600 and 4500 g, and were fully formula fed for at least four weeks before the start of the intervention period. Infants with congenital abnormalities, or with proven or suspected cow's milk allergy, infants derived from multiple births, infants that had received antibiotics less than two weeks before the start of the study, and infants that were fed any pro- or prebiotic formula less than a month before the start of the study, were excluded from the study. After enrolment, infants were randomly allocated to one of two treatment groups: a group receiving an infant formula supplemented with 0.8 g/100 ml galacto-oligosaccharides and fructo-polysaccharides (GFSF-group) and a group receiving a standard infant formula (SF-group). The macronutrient composition of the formulas is shown in table 5.

TABLE 5
Macronutrient composition of the study
formulas (per 100 ml ready to use formula)

| | Carbohydrate mixture- | Standard |
|---|---|---|
| | supplemented formula | formula |
| | (Aptamil 1 with | (Aptamil 1, |
| | GOS/FOS, Milupa) | Milupa) |
| Energy (kcal) | 72 | 72 |
| Protein (g) | 1.5 | 1.5 |
| Carbohydrate (g) | 8.5 | 8.5 |
| Lactose (g) | 7.5 | 7.5 |
| Starch (g) | 1 | 1 |
| Non-digestible oligosaccharides (g) | 0.8 | 0 |
| Galacto-oligosaccharides (g) | 0.72 | 0 |
| Fructo-polysaccharides (g) | 0.08 | 0 |
| Fat (g) | 3.6 | 3.6 |

A group of breast-fed infants was included as a reference group (BF group). Within three days after the start of the study period, after 4 weeks, and at the end of the study period (6 weeks), faecal samples were collected. The study was approved by the medical ethical committees of the four hospitals. Written informed consent was obtained from the parents before the start of the study.

Nucleic acids were isolated from faeces by thawing faecal samples in ice water, followed by a 10× (w/v) dilution in PBS (0.37 M NaCl, 2.7 mM KCl, 8.1 mM Na₂HPO₄ [pH 7.4]) and homogenisation for 10 minutes using a stomacher (IUL Instruments, Barcelona, Spain). Homogenised faeces was stored at −20° C. prior to the actual DNA isolation. The extractions were started by thawing 1 ml of a homogenised faeces sample in ice water, followed by centrifugation for 1 minute at 1100 rpm to remove debris and large particles. Supernatants were transferred to a new tube and centrifuged for 5 minutes at 10000 rpm. Subsequently, the pellets were resuspended in 1 ml TN150 (10 mM Tris-HCl [pH 8.0], 10 mM EDTA) and transferred to sterile tubes containing 0.3 g zirconium beads (diameter 0.1 mm, BioSpec Products, Bartlesville, US). To these suspensions 150 μl of TE-buffered phenol (pH ±7.5) was added and the samples were placed in a mini-bead beater (BioSpec Products, Bartlesville, US), for 3 minutes at 5000 rpm. After bead-beating the samples were immediately cooled on ice, before addition of 150 μl chloroform. Samples were vortexed shortly and centrifuged for 5 minutes at 10000 rpm, upper phases were transferred to clean 2 ml eppendorf tubes and the phenol/chloroform extraction was started. Phenol-chloroform extraction was followed by precipitation of DNA through placement of the samples at −20° C. for at least 30 minutes, after addition of 1 ml ice-cold ethanol (96%) and 50 μl 3 M sodium acetate (pH 5.2). Consecutively, the samples were centrifuged for 20 minutes at 13000 rpm and washed with 500 μl 70% ethanol. After centrifugation for 5 minutes at 13000 rpm, the supernatants were discarded and the pellets were air dried at room temperature. The DNA was resuspended in 100 μl sterile milli-Q and stored at −20° C.

The duplex 5′ nuclease assays are used for the relative quantification of the different Bifidobacterium species in faecal samples. The relative amount of each species is calculated according to Liu et. al. 2002. Briefly, efficiency of each amplification curve was calculated separately, by the formula E=(threshold_{A}/threshold_{B})^{-(Ct,A-Ct,B)}−1. With help of the calculated efficiencies the initial amount of DNA (R₀) is calculated by R₀=threshold/(1+E)^{Ct}. The initial amount of DNA of a Bifidobacterium species can then be divided with the initial amount of DNA of all Bifidobacterium species. Thereafter the obtained ratio's can be normalised with help of the ratio of a monoculture, which is set to 100%.

The total amount of Bifidobacterium was also determined with help of FISH, like earlier described (Langendijk, F. Schut, G. J. Jansen, G. C. Raangs, G. R. Kamphuis, M. H. Wilkinson and G. W. Welling “Quantitative fluorescence in situ hybridisation of Bifido-bacterium spp. with genus-specific 16S rRNA-targeted probes and its application in fecal samples” Appl. Environ. Microbiol. 61(8):3069-75. (1995))

The percentage of the genus Bifidobacterium as a percentage of total bacteria was 75, 47, and 68% in the BF, SF, and GFSF group, respectively, which demonstrates that the GFSF group, fed a mixture of nondigestible carbohydrates, has a more bifidogenic flora, as in the BF group, than in the SF group.

In table 6 the prevalence of each species in the different groups at the beginning as well as at the end of the study is shown. In table 7 the percentage of Bifidobacteria species relative to the total amount of Bifidobacteria is shown.

TABLE 6
Prevalence (in %) of Bifidobacteria species in the faeces of infants
after 6 weeks of feeding with human milk (BF), an infant formula
with a prebiotic mixture (GFSF) or with a standard formula (SF).

| Species | BF | GFSF | SF |
|---|---|---|---|
| B. catenulatum | 80 | 67 | 75 |
| B. adolescentis | 20 | 11 | 50 |
| B. breve | 70 | 78 | 63 |
| B. longum | 50 | 56 | 63 |
| B. bifidum | 10 | 11 | 13 |
| B. angulatum | 30 | 11 | 13 |
| B. infantis | 100 | 100 | 100 |
| B. dentium | 20 | 11 | 13 |

TABLE 7
Percentage of Bifidobacteria species with respect to the total number
of Bifidobacteria in the faeces after a 6 week feeding period.

| | Breast-fed | | GFSF-fed | | SF-fed | |
|---|---|---|---|---|---|---|
| Species | % (sd) | | % (sd) | | % (sd) | |
| B. catenulatum | 1.9 | (1.0) | 1.5 | (3.0) | 9.8 | (12.6) |
| B. adolescentis | 0.3 | (0.9) | 0.1 | (0.2) | 2.9 | (6.0) |
| B. breve | 11.7 | (9.6) | 5.4 | (10.8) | 4.9 | (10.7) |
| B. longum | 7.3 | (13.9) | 5.4 | (10.7) | 6.2 | (9.4) |
| B. bifidum | <0.1 | (0.0) | <0.1 | (0.0) | <0.1 | (0.0) |
| B. angulatum | <0.0 | (0.0) | <0.1 | (0.2) | <0.1 | (0.0) |
| B. infantis | 32.0 | (18.9) | 32.1 | (20.0) | 37.8 | (18.4) |
| B. dentium | <0.1 | (0.0) | <0.1 | (0.0) | <0.1 | (0.0) |

A large variety of Bifidobacterium species is present in the three different groups. Furthermore, a significant decrease in prevalence and amount of B. adolescentis is visible in breast-fed infants and in infants receiving GFSF contrary to infants receiving a standard formula. After 6 weeks of feeding the prevalence and percentage of B. adolescentis is much higher in SF-fed babies than in babies which were GFSF or breast-fed. Analyses of the faecal samples of GFSF infants shows a large variety in the bifidobacterial flora similar to breast-fed infants and stimulation of only one or a few species is not observed. Besides the effect on B. adolescentis the profiles of breast-fed infants and infants receiving GFSF also showed less B. catenulatum (+B. pseudocatenulatum) than the profile of infants receiving a standard formula. B. infantis, and B. longum seems to be predominant in breast-fed infants as well as in infants receiving a standard formula (SF) or a standard formula supplemented with prebiotics (GFSF). Also B. breve was dominant in all three groups, but in the group receiving breast milk B. breve as a % of total bifidobacteria was higher (11.7%) as in the SF (4.9%) and GFSF (5.4%) group.

### Example 3

### Animal Experiments on Allergy

Specific pathogen free male BALB/c mice were obtained from Charles River (Maastricht, the Netherlands). Food and water was provided ad libitum and the mice were used when 6-9 weeks of age. All experiments were approved by the animal ethics committee of the University of Utrecht, The Netherlands.

Ovalbumin (grade V) and acetyl-β-methylcholine chloride (methacholine) were purchased from Sigma Chemical Co. (St. Louis, Mo., USA). Aluminium hydroxide (Alumlmject) was purchased from Pierce (Rockford, Ill., USA).

Mice were sensitised by two i.p. injections with 10 μg ovalbumin adsorbed onto 2.25 mg aluminium hydroxide in 100 μl saline or saline alone on days 0 and 7. Mice were challenged on days 35, 38, and 41 by inhalation of ovalbumin aerosols in a plexiglass exposure chamber for 20 minutes. The aerosols were generated by nebulising an ovalbumin solution (10 mg/ml) in saline using a Pari LC Star nebulizer (Pari respiratory Equipment, Richmond, Va., USA).

Mice were treated daily with 1×10e9 (CFU) Bifidobacterium breve and 25 mg of a mixture of galactooligosaccharides and fructopolysaccharides (9:1) orally via gavage (0.2 ml, physiological salt solution) starting at day 28 upto the end of the experiment (i.e. day 42). As a control 0.2 ml physiological salt solution was administered via gavage.

Airway responsiveness to inhaled nebulised methacholine was determined 24 hours after the final aerosol challenge, in conscious, unrestrained mice using whole body plethysmo-graphy (BUXCO, EMKA, Paris, France). The airway response was expressed as enhanced pause (PenH).

Statistical Analysis: The airway response curves to methacholine were statistically analysed by a general linear model or repeated measurements followed by post-hoc comparison between groups. Cell counts were statistically analysed using the Mann-Whitney U test (Siegel, S., Castellan Jr. N J, 1988, “Nonparametric statistics for the behavioural sciences” 2^{nd} ed. McGraw Hill Book Company, New York, USA). All other analyses were performed using Student's t-test (Abramowitz, M., Stegun, I. A., 1972, “Handbook of mathematical functions” Dover publications, Inc. New York, USA). A probability value of p<0.05 was considered as statistically significant.

Results on airway hyperresponsiveness: Measurements on airway hyperresponsiveness show that compared to control the mice receiving the B. breve+a mixture of galactooligosaccharides and fructopolysaccharides show a statistically reduced airway hyperresponsiveness, indicative of a lowered asthmatic reaction.

In FIG. 2 the airway hyperresponsiveness is plotted as relative PenH (enhanced pause) versus the metacholine concentration for mice receiving a combination of B. Breve+a mixture of GOS/FOS and a control group of mice receiving saline instead. The plotted values of relative PenH are obtained after subtraction of the blank values obtained for mice not ovalbumin-sensitised and normalisation to the value obtained for the control group at the highest concentration of metacholine.

The compositions of all following examples may additionally contain minerals, trace elements and vitamins, choline, taurine, carnitine, and/or myo-inositol or mixtures thereof, as known in the art and in accordance with international guidelines. Furthermore, organic acids, flavours and or colorants may or may not be present.

### Example 4

An infant milk formula containing per 100 ml final product (and per 13.1 g powder):

| | | |
|---|---|---|
| 8 energy % protein | 1.4 | g (casein whey mixture) |
| 45 energy % digestible | 7.5 | g |
| carbohydrates | | |
| 47 energy % fat | 3.5 | g |
| GOS (90% | 0.4 | g |
| galacto-oligosaccharides, Borculo | | |
| Domo NL)/polyfructose, (10% | | |
| inulin, Raftilin HP, Orafti BE) | | |
| B. breve: | 1.3 × 10⁸ | cfu |

### Example 5

An infant milk formula containing per 100 ml final product (and per 14 g powder):

| | | |
|---|---|---|
| 10 energy % protein | 1.8 | g (casein whey mixture) |
| 46 energy % digestible | 8.0 | g |
| carbohydrates | | |
| 44 energy % fat | 3.4 | g |
| GOS/polyfructose (see ex. 4) | 0.4 | g |
| B. breve | 1.4 × 10⁸ | cfu |

### Example 6

An infant milk formula containing per 100 ml final product (and per 16.1 g powder):

| | | |
|---|---|---|
| 10 energy % protein | 1.9 | g (casein whey mixture) |
| 51 energy % digestible | 9.9 | g |
| carbohydrates | | |
| 39 energy % fat | 3.3 | g |
| FOS (Raftilin, | 0.4 | g |
| Orafti)/galactomannan 9/1 | | |
| B. breve | 1.6 × 10⁸ | cfu |

### Example 7

An infant milk formula containing per 100 ml final product (and per 13 g powder):

| | | |
|---|---|---|
| 10 energy % protein | 1.6 | g (hydrolysed whey protein) |
| equivalent | | |
| 42 energy % digestible | 7.1 | g |
| carbohydrates | | |
| 48 energy % fat | 3.6 | g |
| sialyl oligosaccharides, | 0.4 | g |
| indigestible maltodextrins 9/1 | | |
| B. breve | 6.5 × 10⁸ | cfu/g |

### Example 8

An infant milk formula containing per 100 ml final product (and per 15 g powder):

| | | |
|---|---|---|
| 10 energy % protein | 1.8 | g (hydrolysed whey protein) |
| equivalent | | |
| 42 energy % digestible | 8.6 | g |
| carbohydrates | | |
| 44 energy % fat | 3.6 | g |
| fuco-oligosaccharides(from | 0.4 | g |
| algal fucoidan), | | |
| galactomannan 8/2 | | |
| B. breve | 7.5 × 10⁸ | cfu |

### Example 9

An infant milk formula containing per 100 ml final product (and per 15.1 g powder):

| | | |
|---|---|---|
| 10 energy % protein | 1.8 | g (hydrolysed whey protein) |
| equivalent | | |
| 42 energy % digestible | 8.6 | g |
| carbohydrates | | |
| 44 energy % fat | 3.6 | g |
| manno-oligosaccharides, | 0.4 | g |
| arabinogalactan 9/1 | | |
| B. breve | 1.5 × 10⁸ | cfu |

### Example 10

An infant milk formula containing per 100 ml final product (and per 15.2 g powder):

| | | |
|---|---|---|
| 10 energy % protein | 1.7 | g (hydrolysed whey protein) |
| 48 energy % digestible | 8.4 | g |
| carbohydrates | | |
| 42 energy % fat | 3.3 | g |
| GOS/galactoruonic oligo- | 1.0 | g |
| saccharides//polyfructose | | |
| 7/2/1 | | |
| B. breve | 7.5 × 10⁸ | cfu |

### Example 11

An infant milk formula containing per 100 ml final product (and per 15.8 g powder):

| | | |
|---|---|---|
| 11 energy % protein | 1.9 | g (hydrolysed whey protein) |
| 48 energy % digestible | 8.7 | g |
| carbohydrates | | |
| 41 energy % fat | 3.3 | g |
| xylooligosaccharides/galactan | 0.8 | g |
| 9/1 | | |
| B. breve | 8 × 10⁸ | cfu |

### Example 12

An infant milk formula containing per 100 ml final product (and per 15 g powder):

| 10 energy % protein | 1.7 | g (casein whey mixture) |
|---|---|---|
| 48 energy % digestible | 8.1 | g |
| carbohydrates | | |
| 42 energy % fat | 3.1 | g |
| GOS/polyfructose 9/1 | 0.8 | g |
| Galactomannan | 0.42 | |
| B. breve | 1.5 × 10⁸ | cfu |

### Example 13

An infant milk formula containing per 100 ml final product (and per 15.9 g powder):

| | | |
|---|---|---|
| 13 energy % protein | 2.2 | g (casein whey mixture) |
| 49 energy % digestible | 8.6 | g |
| carbohydrates | | |
| 37 energy % fat | 3.0 | g |
| GOS/polyfructose 9/1 | 0.8 | g |
| Galactomannan | 0.4 | g |
| B. breve | 1.6 × 10⁸ | cfu |

### Example 14

An infant milk formula containing per 100 ml final product (and per 13.5 g powder):

| | | |
|---|---|---|
| 9 energy % protein equivalent | 1.5 | g (hydrolysed whey protein) |
| 42 energy % digestible | 6.9 | g |
| carbohydrates | | |
| 49 energy % fat | 3.6 | g |
| GOS/polyfructose/ | 0.8 | g |
| sialyllactose 7/2/1 | | |
| B. breve | 1.4 × 10⁸ | cfu |

### Example 15

An infant milk formula containing per 100 ml final product (and per 13.7 g powder):

| | | |
|---|---|---|
| 9 energy % protein equivalent | 1.4 | g (free amino acids) |
| 44 energy % digestible carbohydrates | 7.1 | g |
| 47 energy % fat | 3.4 | g |
| GOS/polyfructose 6/4 | 0.8 | g |
| B. breve | 1.4 × 10⁸ | cfu |

### Example 16

An infant formula containing per 100 ml final product (and per 13.5 g powder):

| | | |
|---|---|---|
| 11 energy % protein | 1.8 | g (soy protein) |
| 40 energy % digestible carbohydrates | 6.7 | g |
| 49 energy % fat | 3.6 | g |
| GOS/galacto-ogosaccharides/polyfructose | 0.8 | g |
| 8/1/1 | | |
| B. breve | 1.4 × 10⁸ | cfu |

### Example 17

An infant formula containing per 100 ml final product (and per 15.1 g powder):

| | | |
|---|---|---|
| 12 energy % protein | 2.2 | g (soy protein) |
| 43 energy % digestible carbohydrates | 7.7 | g |
| 45 energy % fat | 3.6 | g |
| FOS/galactan 9/1 | 0.8 | g |
| B. breve | 1.5 × 10⁸ | cfu |

### Example 18

An infant formula containing per 100 ml (and 16.5 g powder)

| | | |
|---|---|---|
| 13 energy % protein | 2.0 | g (hydrolysed whey) |
| 57 energy % digestible carbohydrates | 8.6 | g |
| 30 energy % fat | 2.0 | g |
| GOS/polyfructose 9/1 | 1.0 | |
| Soy polysaccharides | 0.5 | |
| B. breve | 1.5 × 10⁹ | cfu |

### Example 19

A milk-based product containing per 100 ml

| | | |
|---|---|---|
| 14 energy % protein | 2.5 | g (cow's milk protein) |
| 43 energy % digestible carbohydrates | 7.5 | g |
| 43 energy % fat | 3.4 | g |
| GOS/polyfructose 7/3 | 1.5 | g |
| B. breve | 3 × 10⁸ | cfu |

### Example 20

An infant formula containing per 100 ml (and 15.4 g powder)

| | | |
|---|---|---|
| 11 energy % protein | 2.0 | g (hydrolysed collagen and soy protein) |
| 46 energy % digestible | 8.6 | g |
| carbohydrates | | |
| 43 energy % fat | 3.6 | g |
| GOS/polyfructose 3/1 | 0.4 | g |
| B. breve | 6 × 10⁸ | cfu |

### Example 21

A supplement: 3 g powder to be added to 100 ml milk: containing:

| | | |
|---|---|---|
| 28 energy % protein | 0.7 | g (casein whey mixture) |
| 72 energy % digestible carbohydrates | 2.0 | g |
| GOS/polyfructose 65/35 | 0.3 | g |
| B. breve | 3 × 10⁹ | cfu |

### Example 22

A supplement containing: 0.4-0.8 g to be added to 100 ml milk: per g:

| | | |
|---|---|---|
| 0.26 | g | galactomannan, |
| 0.44 | g | digestible carbohydrates |
| 0.3 | g | GOS/polyfructose 85/15 |
| 1.0 × 10⁹ | cfu | B. breve |

### Example 23

A supplement containing per 100 ml

| 100% energy digestible carbohydrates | 2.2 | g |
|---|---|---|
| minerals: | K, Na, Cl, | |
| osmolarity | 261 | mOsm/l |
| GOS/polyfructose 55/45 | 0.4 | g |
| B. breve | 1 × 10⁹ | cfu |

### Example 24

An infant nutrition containing per 100 g (85 g to be added to 240 ml milk)

| | | |
|---|---|---|
| 4 energy % protein | 4.7 | g (cow's milk protein) |
| 53 energy % digestible carbohydrates | 68 | g |
| 43 energy % fat | 24.6 | g |
| GOS/polyfructose 9/1 | 0.8 | g |
| B. breve | 1.2 × 10⁹ | cfu |

### Example 25

An infant nutrition (tube feed): per 100 ml

| | | |
|---|---|---|
| 9 energy % protein | 3.4 | g (casein) |
| 50 energy % carbohydrates | 18.8 | g |
| 41 energy % fat | 8 | g |
| GOS/polyfructose 7/3 | 0.4 | g |
| B. breve | 5 × 10⁸ | cfu |

### Example 26

An infant nutrition containing per 100 ml product

| | | |
|---|---|---|
| 11 energy % protein | 2.8 | g (casein) |
| 49 energy % carbohydrates | 12.3 | g |
| 40 energy % fat | 4.4 | g |
| GOS/polyfructose 85/15 | 0.8 | g |
| B. breve | 5 × 10⁸ | cfu |

### Example 27

An infant nutrition composed of rice flour containing per 100 g dry product: (4-7 spoons to be added to 200 ml warm infant formula, follow-on formula, toddler's milk or cow's milk)

| | | |
|---|---|---|
| 7.4 | g | protein (vegetable) |
| 83 | g | carbohydrates |
| 0.5 | g | fat |
| 3 | g | dietary fibre including 1.5 g GOS/polyfructose 9/1 |
| 1 × 10¹⁰ | cfu | B. breve |

### Example 28

An infant nutrition composed of precooked flakes (wheat, rye, rice, barley, maize, oat, buckwheat) containing per 100 g dry product. (5-7 spoons to be added to 250 ml warm infant formula, follow-on formula, toddler's milk or cow's milk)

| | | |
|---|---|---|
| 9.5 | g | protein (vegetable) |
| 74 | g | carbohydrates |
| 2.0 | g | fat |
| 3 | g | dietary fibre including 1.5 g GOS/polyfructose 8/2 |
| 2 × 10¹⁰ | cfu | B. breve |

### Example 29

An infant nutrition composed of homogenised vegetables or fruit, containing per 100 ml

| | | |
|---|---|---|
| GOS/polyfructose 75/25 | 2.0 | g |
| B. breve | 2 × 10⁹ | cfu |

1. A method for normalizing the Bifidobacterium species composition in the gastro-intestinal tract of non- or partially breast-fed infants to the composition found in breast-fed infants, the method comprising administering to the non- or partially breast-fed infant a composition comprising Bifidobacterium breve and a mixture of at least two non-digestible soluble carbohydrate components A and B, the carbohydrate component A being present in an amount of from 5 to 95% by weight of the sum of carbohydrate components A and B, at least 50% of the total non-digestible soluble carbohydrates being selected from disaccharides to eicosasaccharides, components A and B differing either:
(i) in the (average) number of monosaccharide units of the carbohydrate, component A having an average chain length which is at least 5 monosaccharide units lower than the average chain length of component B, or
(ii) in the structure of the monosaccharide units of the carbohydrate, or
(iii) both.

2. The method according to claim 1, wherein the carbohydrate component A has a different structure from the carbohydrate component B.

3. The method according to claim 1, wherein the carbohydrate components A and B differ in the (average) number of monosaccharides units, component A being selected from indigestible monosaccharides up to hexasaccharides of the same carbohydrate structure, and component B being selected from indigestible heptasaccharides and higher polysaccharides of the same carbohydrate structure.

4. The method according to claim 1, wherein the carbohydrate component A comprises 95 to 60 wt % and the carbohydrate B comprises 5 to 40 wt %, with A+B=100 wt %.

5. The method according to claim 1, wherein 60 wt % to 100 wt % of carbohydrate components A belong to the group of galacto-oligosaccharides.

6. The method according to claim 5, wherein 80 wt % to 100 wt % of carbohydrate components A belong to the group of galacto-oligosaccharides.

7. The method according to claim 1, wherein 60 wt % to 100 wt % of carbohydrate components B belong to the group of fructo-polysaccharides, including inulin.

8. The method according to claim 7, wherein 80 wt % to 100 wt % of carbohydrate components B belong to the group of fructo-polysaccharides, including inulin.

9. The method according to claim 1, comprising 10⁷ to 10¹¹ cfu Bifidobacterium breve per g of total non-digestible soluble carbohydrate.

10. The method according to claim 9, comprising 10⁸ to 10¹⁰ cfu Bifidobacterium breve per g of total non-digestible soluble carbohydrate.

11. The method according to claim 1, further comprising digestible carbohydrate, a lipid source, and a protein source.

13. A method for the prevention or treatment of one or more immune disorders, comprising administering to a subject in need thereof a composition comprising Bifidobacterium breve and a mixture of at least two non-digestible soluble carbohydrate components A and B, the carbohydrate component A being present in an amount of from 5 to 95% by weight of the sum of carbohydrate components A and B, at least 50% of the total non-digestible soluble carbohydrates being selected from disaccharides to eicosasaccharides, components A and B differing either:
(i) in the (average) number of monosaccharide units of the carbohydrate, component A having an average chain length which is at least 5 monosaccharide units lower than the average chain length of component B, or
(ii) in the structure of the monosaccharide units of the carbohydrate, or
(iii) both.

14. The method according to claim 13, wherein the immune disorders are selected from allergy, atopy, allergic rhinitis, food hypersensitivity, atopic dermatitis, eczema and asthma.

15. The method to claim 13, wherein the immune disorders are selected from diarrhoea and viral diarrhoea.

16. A preparation comprising Bifidobacterium breve and a mixture of at least two non-digestible soluble carbohydrate components A and B, the carbohydrate component A being present in an amount of from 5 to 95% by weight of the sum of carbohydrate components A and B, at least 50% of the total non-digestible soluble carbohydrates being selected from disaccharides to eicosasaccharides, components A and B differing either:
(i) in the (average) number of monosaccharide units of the carbohydrate, component A having an average chain length which is at least 5 monosaccharide units lower than the average chain length of component B, or
(ii) in the structure of the monosaccharide units of the carbohydrate, or
(iii) both.

17. A method for preventing and/or treating energy malabsorption, comprising administering to a subject in need thereof a composition according to claim 16.

18. A method for inhibiting the infiltration of eosinophils, neutrophils and mononuclear cells in allergic lesions, inhibiting the Th2 type immune response and/or stimulating the Th1 mediated immune response, the method comprising administering to a subject in need thereof a composition according to claim 16.

19. Oligonucleotides comprising SEQ ID selected from SEQ ID No 1, SEQ ID No 2, SEQ ID No 4, SEQ ID No 5, SEQ ID No 7, SEQ ID No 8, SEQ ID No 10, SEQ ID No 11, SEQ ID No 13, SEQ ID No 14, SEQ ID No 16, SEQ ID No 17, SEQ ID No 19, SEQ ID No 20, SEQ ID No 22, SEQ ID No 23, SEQ ID No 25, SEQ ID No 26, and sequences complementary thereto.

20. A method of species-specifically detecting species of the genus Bifidobacterium found in human, comprising:
(A) contacting a sample with an oligonucleotide probe in a hybridising solution, wherein the probe is selected from the group consisting of:
1) a labelled oligonucleotide which specifically hybridises to B. adolescentis DNA represented by SEQ ID No 3 or a sequence complementary thereto;
2) a labelled oligonucleotide which specifically hybridises to B. angulatum DNA represented by SEQ ID No 6 or a sequence complementary thereto;
3) a labelled oligonucleotide which specifically hybridises to B. bifidum DNA represented by SEQ ID No 9 or a sequence complementary thereto;
4) a labelled oligonucleotide which specifically hybridises to B. breve DNA represented by SEQ ID No 12 or a sequence complementary thereto;
5) a labelled oligonucleotide which specifically hybridises to B. catenulatum DNA represented by SEQ ID No 15 or a sequence complementary thereto;
6) a labelled oligonucleotide which specifically hybridises to B. dentium DNA represented by SEQ ID No 18 or a sequence complementary thereto;
7) a labelled oligonucleotide which specifically hybridises to B. infantis DNA represented by SEQ ID No 21 or a sequence complementary thereto;
8) a labelled oligonucleotide which specifically hybridises to B. longum DNA represented by SEQ ID No 24 or a sequence complementary thereto;
9) a labelled oligonucleotide which specifically hybridises to all Bifidobacterium DNA represented by SEQ ID No 27 or a sequence complementary thereto, and
(B) determining whether the probe hybridises to nucleic acids in the sample so as to detect whether the species of the genus is present in the sample.
